(19) United States
(12) Patent Application Publication
(10) Pub. No.: US 2025/0375760 A1
(43) Pub. Date: Dec. 11, 2025

(54) CATALYST FOR EXTRACTING HIGH PURITY HYDROGEN FROM ORGANIC HYDROGEN CARRIER AND METHOD OF PREPARING SAME

(71) Applicant: POSTECH RESEARCH AND BUSINESS DEVELOPMENT FOUNDATION, Pohang-si (KR)

(72) Inventors: Chang Won YOON, Pohang-si (KR); Byeong Jo SHIM, Pohang-si (KR); Seungmok HAN, Pohang-si (KR)

(73) Assignee: POSTECH RESEARCH AND BUSINESS DEVELOPMENT FOUNDATION, Pohang-si (KR)

(21) Appl. No.: 19/028,675

(22) Filed: Jan. 17, 2025

(30) Foreign Application Priority Data
Jun. 7, 2024 (KR) 10-2024-0074401
Oct. 22, 2024 (KR) 10-2024-0145178

(51) Int. Cl. B01J 37/02; B01J 23/42; B01J 27/02; B01J 27/16; B01J 35/61; B01J 35/63; B01J 35/64; B01J 37/04; B01J 37/08; C01B 3/26; C07C 5/32

(52) U.S. Cl. CPC B01J 37/024; B01J 23/42; B01J 27/02; B01J 27/16; B01J 35/613; B01J 35/633; B01J 35/647; B01J 37/04; B01J 37/08; C01B 3/26; C07C 5/325; C01B 2203/0277; C01B 2203/107; C01B 2203/1082; C01B 2203/1252; C07C 2523/42

(57) ABSTRACT

Disclosed are catalyst for extracting high purity hydrogen from organic hydrogen carrier and catalyst composite of preparing same. In detail, a catalyst composite comprising: a support comprising a metal oxide doped with phosphorus(P); and a catalyst comprising platinum group nanoparticle and sulfur(S) and supported on the support, wherein the platinum group nanoparticle may comprise a platinum group element, and the sulfur(S) may be doped on a part or all of a surface of the platinum group nanoparticle. The present disclosure enables easily and quickly support metal nanoparticles on powder and bead-structured supports using wet-impregnation.

### CROSS REFERENCE TO RELATED APPLICATION

The present application claims priority to Korean Patent Application No. 10-2024-0074401, filed Jun. 7, 2024, and Korean Patent Application No. 10-2024-0145178, filed Oct. 22, 2024, the entire contents of which are incorporated herein for all purposes by these references.

### BACKGROUND OF THE DISCLOSURE

### 1. Field of the Disclosure

The present disclosure relates to catalyst for extracting high purity hydrogen from organic hydrogen carrier and method of preparing same.

### 2. Description of the Related Art

Monobenzyltoluene, one of the liquid organic hydrogen carriers, is attracting attention as a potent hydrogen carrier for the introduction of large-capacity hydrogen overseas due to its high hydrogen storage by weight (6.22 wt. %), high energy density by volume (54.5 kg-H₂/m³), and liquid characteristics at room temperature and pressure. In addition, the monobenzyltoluene has similar physical properties to existing petroleum fuels, so it is easy to expand economic feasibility by using the existing petroleum infrastructure as it is.

Various liquid organic hydrogen carriers are currently being developed. Thermochemical reaction equation for hydrogen extraction through decomposition of monobenzyltoluene, which is currently being actively researched and developed domestically and internationally, is C₁₄H₂₆→C₁₄H₁₄+6H₂ (ΔH=63.5 KJ/mol). This reaction is an endothermic reaction, and hydrogen can be extracted from a hydrogen storage medium using an external heat source. For this purpose, high temperature reaction conditions of 320° C. or higher at atmospheric pressure and the corresponding catalyst are required. The conventional technology for extracting hydrogen from thermochemical hydrogenated monobenzyltoluene is a relatively recently developed liquid organic hydrogen carrier, and the development of hydrogen extraction catalysts for the corresponding compound has hardly been done. The conventional technology currently requires a reaction condition of high temperature, and the need for a catalyst that can generate a hydrogen extraction reaction efficiently and stably at that temperature is required. However, the reactivity of the catalyst is reduced due to the flow rate of the liquid during the reaction and the coke caused by the reaction, which causes a rapid decrease in the catalyst efficiency, lowers the hydrogen production amount, and shortens the life of the catalyst. In addition, since Pt (platinum) metal is used as the active metal of the catalyst, it is essential to reduce the content of expensive platinum.

Therefore, an economical and efficient catalyst development that can lower the reaction temperature and minimize the content of precious metals is required.

### SUMMARY OF THE DISCLOSURE

The purpose of the present disclosure is to solve the above problems, and to increase the degree of dispersion of platinum particles by doping phosphorus on a support to increase the efficiency of hydrogen extraction, and to provide a catalyst in which sulfur is selectively adsorbed on the platinum particles by doping sulfur on a platinum catalyst using a phosphorus-doped support.

In addition, the other purpose of the present disclosure is to provide a catalyst that makes it possible to control platinum particles with a very small amount of sulfur and maximizes stability.

In addition, the other purpose of the present disclosure is to provide a catalyst that can show high activity under a reaction temperature of 320° C. and has improved stability at that temperature.

In addition, the other purpose of the present disclosure is to provide a method of preparing a catalyst for hydrogen extraction using an impregnation method.

One aspect of the present disclosure provides catalyst composite, the catalyst composite comprising: a support comprising a metal oxide doped with phosphorus(P); and a catalyst comprising a platinum group nanoparticle and sulfur(S) and supported on the support, wherein the platinum group nanoparticle may comprise a platinum group element, and the sulfur(S) may be doped on a part or all of a surface of the platinum group nanoparticle.

In addition, the doped phosphorus may be located at an acidic site of the metal oxide.

In addition, the phosphorus may be doped onto the metal oxide in the form of a phosphate group.

In addition, the platinum group element may be a partially positively charged due to the sulfur.

In addition, the phosphate group may suppress the doping of the sulfur onto the metal oxide.

In addition, the metal oxide may comprise at least one selected from the group consisting of alumina (Al₂O₃), cerium oxide (CeO₂), magnesium oxide (MgO), carbon composite (C), silica (SiO₂) and titania (TiO₂).

In addition, the catalyst composite may comprise 0.01 to 2 parts by weight of the phosphorus based on 100 parts by weight of the metal oxide.

In addition, the platinum group element may comprise at least one selected from the group consisting of Pt, Pd, Ru, Rh, Os and Ir.

In addition, the size of the platinum group nanoparticle may be in a range of 0.5 to 10 nm.

In addition, the catalyst composite may comprise 0.1 to 2 parts by weight of the platinum group nanoparticle based on 100 parts by weight of the metal oxide.

In addition, the catalyst composite may comprise 0.01 to 0.5 parts by weight of the sulfur based on 100 parts by weight of the metal oxide.

In addition, the catalyst composite may be used to extract hydrogen by dehydrogenating an organic hydrogen carrier.

Another aspect of the present disclosure provides a method of preparing a catalyst composite, the method comprising: (a) stirring a mixture comprising a metal oxide, a phosphorus(P) precursor, and a first solvent and drying; (b) preparing a support comprising a phosphorus(P)-doped metal oxide by heat-treating the resultant of step (a) thus preparing a support comprising a phosphorus(P)-doped metal oxide; (c) stirring a mixture comprising the support, a platinum group element precursor, and a second solvent and drying; (d) supporting platinum group nanoparticle comprising a platinum group element on the support by heat-treating the resultant of step (c); (e) stirring a mixture comprising the support on which the platinum group nanoparticles are supported, a sulfur precursor, and a third solvent and drying; and (f) preparing a catalyst composite by heat-treating the resultant of step (e).

In addition, the catalyst composite may comprise a support comprising a metal oxide doped with phosphorus(P); and a catalyst comprising platinum group nanoparticle and sulfur(S) and supported on the support, wherein the platinum group nanoparticle comprises a platinum group element, and the sulfur(S) is doped onto a part or all of a surface of the platinum group nanoparticle.

In addition, the heat treatments of steps (b), (d) and (f) may be carried out at a temperature range of 400 to 600° C. respectively.

In addition, the first solvent, the second solvent, and the third solvent may comprise water respectively.

In addition, the phosphorus precursor may comprise at least one selected from the group consisting of (NH₄)₂HPO₄, NH₄H₂PO₄, phosphoric acid (H₃PO₄), phytic acid (C₆H₁₈O₂₄P₆), phosphine (PH₃), teriethoxyphosphine (C₆H₁₅O₃P) and triphenylphosphine ((C₆H₅)₃P), the platinum group element precursor may comprise at least one selected from the group consisting of H₂PtCl₆, Pt(NO₃)₂, Pt(NH₃)₂ (NO₂)₂ and Pt(NH₃)₄ (OH)₂ and the sulfur precursor may comprise at least one selected from the group consisting of (NH₄)₂SO₄, sulfuric acid (H₂SO₄), thiourea ((NH₂)₂CS), thioamide, hydrogen sulfide (H₂S) and sodium thiosulfate (Na₂S₂O₃).

Another aspect of the present disclosure provides a method of extracting hydrogen, the method comprising: producing a compound represented by structural formula 2 and hydrogen by using a compound represented by structural formula 1 and a catalyst composite, as in reaction scheme 1.

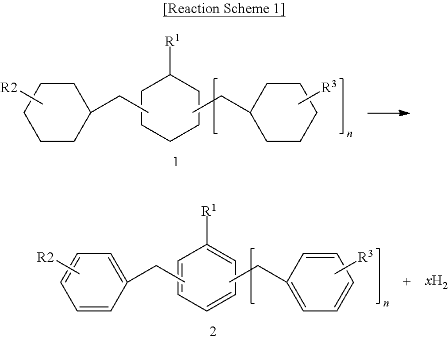

in the reaction scheme 1, R¹ is a hydrogen atom or a C1 to C3 alkyl group, R² is a hydrogen atom or a C1 to C3 alkyl group, R³ is a hydrogen atom or a C1 to C3 alkyl group, n is any one of integers 0 to 2, and x is any one of integers 6 to 12.

In addition, in the reaction scheme 1, R¹ is a methyl group, R² is a hydrogen atom, R³ is a hydrogen atom, n is any one of integers 0 to 2, and x may be any one of integers 6 to 12.

In addition, the catalyst composite may comprise a support comprising a metal oxide doped with phosphorus(P); and a catalyst comprising platinum group nanoparticle and sulfur(S) and supported on the support, wherein the platinum group nanoparticle comprises a platinum group element, and the sulfur(S) may be doped on a part or all of a surface of the platinum group nanoparticle.

According to the present disclosure, metal nanoparticles can be easily and quickly supported on powder and bead-structured supports using wet-impregnation.

In addition according to the present disclosure, it is possible to induce a reduction in catalyst cost through the development of a low content of precious metal in catalyst.

In addition according to the present disclosure, it is possible to improve efficiency through selective adsorption of sulfur on platinum metal.

In addition according to the present disclosure can provide a commercially applicable structure bead type catalyst.

In addition the catalyst composite of the present disclosure can extract hydrogen with a purity of fuel cell grade from a hydrogen storage medium.

### BRIEF DESCRIPTION OF THE DRAWINGS

Since the accompanying drawings are intended to illustrate exemplary embodiments of the present disclosure, the technical ideas of the present invention should not be construed to be limited by the accompanying drawings.

FIG. 1A shows an image of a synthesized catalyst composite of the present disclosure.

FIG. 1B shows a schematic diagram of a thermochemical liquid organic hydrogen carrier hydrogen extraction system of the present disclosure.

FIG. 2A shows a TEM image of Comparative Example 1.

FIG. 2B shows a TEM image of Comparative Example 2.

FIG. 2C shows a TEM image of Comparative Example 3.

FIG. 2D shows a TEM image of Example 1 of the present disclosure.

FIG. 2E shows a TEM image of Example 2 of the present disclosure.

FIG. 3A shows the hydrogen extraction efficiency of Comparative Examples 1 to 4 and Examples 1 and 2 of the present disclosure.

FIG. 3B shows the turnover frequency and deactivation rate of Comparative Examples 1 to 3 and Examples 1 and 2 of the present disclosure.

FIG. 3C shows the hydrogen purity of Comparative Examples 1 to 3 and Examples 1 and 2 of the present disclosure.

FIG. 3D shows the H₂-generation rate of Comparative Examples 1 to 3 and Examples 1 and 2 of the present disclosure.

FIGS. 4A and 4C show the H₂-TPR spectra of bare Al₂O₃, 0.9P—Al₂O₃, Comparative Examples 1 to 3 and Examples 1 and 2 of the present disclosure.

FIGS. 4B and 4D show the NH₃-TPD spectra of bare Al₂O₃, 0.9P—Al₂O₃, Comparative Examples 1 to 3 and Examples 1 and 2 of the present disclosure.

FIGS. 5A to 5C show the XPS spectra of Al 2p and Pt 4f for Comparative Examples 1 to 3 and Examples 1 and 2 of the present disclosure.

FIG. 6A shows the ratio of Pt⁰ and Pt^{δ+} of Comparative Examples 1 to 3 and Examples 1 and 2 of the present disclosure.

FIG. 6B shows the Pt 4d XPS spectra of Comparative Examples 1 to 3 and Examples 1 and 2 of the present disclosure.

FIG. 6C shows the XPS spectra of P 2p (top) in the catalysts of Comparative Examples 2 and 3 and Examples 1 and 2 of the present disclosure and S 2p (bottom) in the catalysts of Examples 1 and 2 of the present disclosure.

FIG. 7A shows the DRIFT spectra of CO adsorbed on the catalysts of Comparative Examples 1 to 3 and Examples 1 and 2 of the present disclosure.

FIG. 7B shows the quantification of Pt site types for the catalysts of Comparative Examples 1 to 3 and Examples 1 and 2 of the present disclosure.

FIG. 8 shows the O₂-TPO analysis of Comparative Examples 1 to 3 and Examples 1 and 2 of the present disclosure.

FIG. 9 shows the dehydrogenation of methylcyclohexane (MCH) of Example 2 of the present disclosure.

FIG. 10 shows the metal surface area of catalysts of Comparative Examples 1 to 3 and Examples 1 and 2 of the present disclosure.

FIG. 11 shows the ³¹P solid-state NMR spectroscopy results of alumina and 0.9P—Al₂O₃.

### DESCRIPTION OF THE PREFERRED EXAMPLES

Herein after, embodiments of the present disclosure will be described in detail with reference to the accompanying drawings in such a manner that the ordinarily skilled in the art can easily implement the embodiments of the present disclosure.

The description given below is not intended to limit the present disclosure to specific embodiments. In relation to describing the present disclosure, when the detailed description of the relevant known technology is determined to unnecessarily obscure the gist of the present disclosure, the detailed description may be omitted.

The terminology used herein is for the purpose of describing particular embodiments only and is not intended to limit the scope of the present disclosure. As used herein, the singular forms “a”, “an”, and “the” are intended to comprise the plural forms as well unless the context clearly indicates otherwise. It will be further understood that the terms “comprise” or “have” when used in the present disclosure specify the presence of stated features, integers, steps, operations, elements and/or combinations thereof, but do not preclude the presence or addition of one or more other features, integers, steps, operations, elements, and/or combinations thereof.

Terms comprising ordinal numbers used in the specification, “first”, “second”, etc. can be used to discriminate one component from another component, but the order or priority of the components is not limited by the terms unless specifically stated. These terms are used only for the purpose of distinguishing a component from another component. For example, without departing from the scope of the present disclosure, a first component may be referred to as a second component, and a second component may be also referred to as a first component.

In addition, when it is mentioned that a component is “formed” or “stacked” on another component, it should be understood such that one component may be directly attached to or directly stacked on the front surface or one surface of the other component, or an additional component may be disposed between them.

Hereinafter, the embodiment of the present disclosure shall be explained with reference to the attached drawing, and in describing it by reference to the accompanying drawing, the same or corresponding components shall be given the same figure number and the duplicate description thereof shall be omitted.

The catalyst for extracting high purity hydrogen from organic hydrogen carrier and method of preparing same will be described in detail. However, those are described as examples, and the present disclosure is not limited thereto and is only defined by the scope of the appended claims.

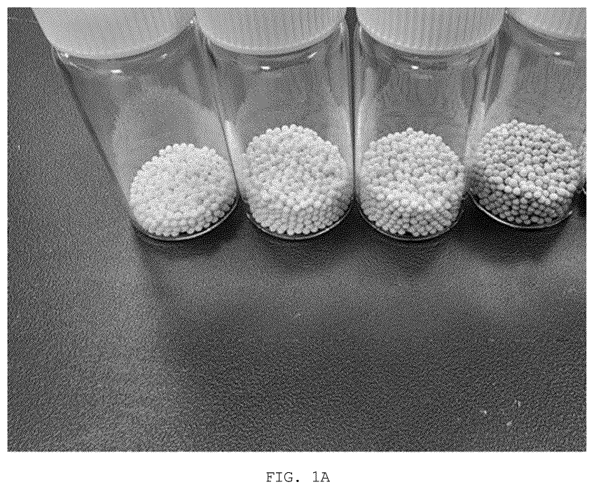

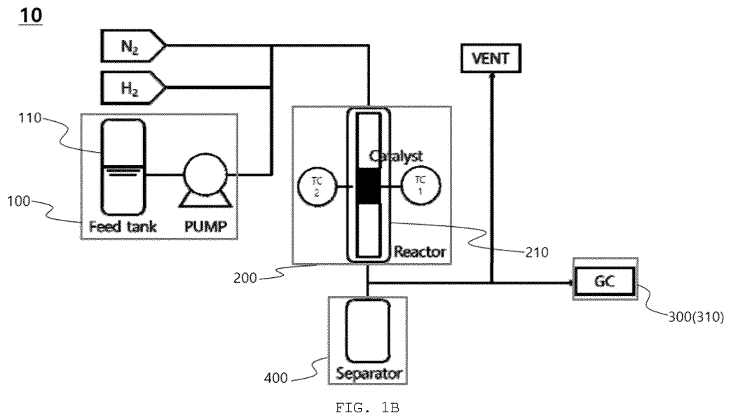

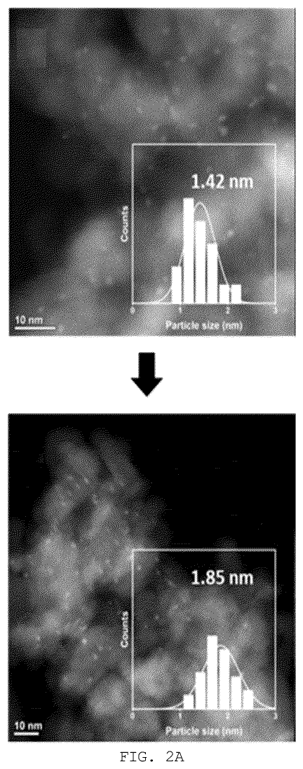

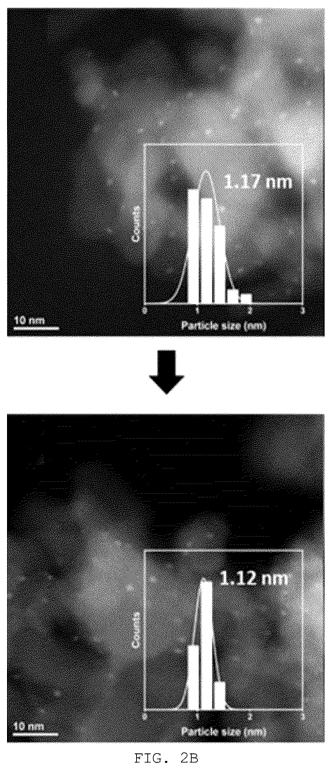

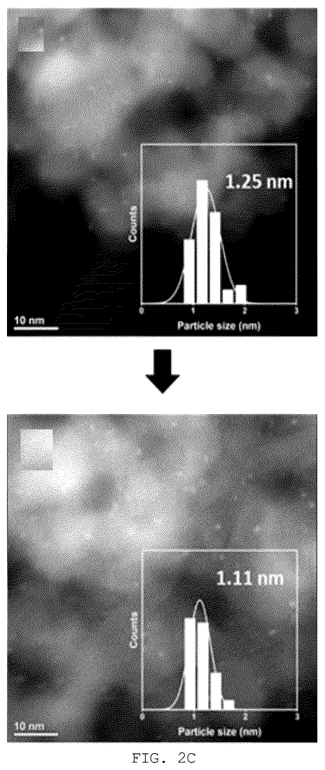

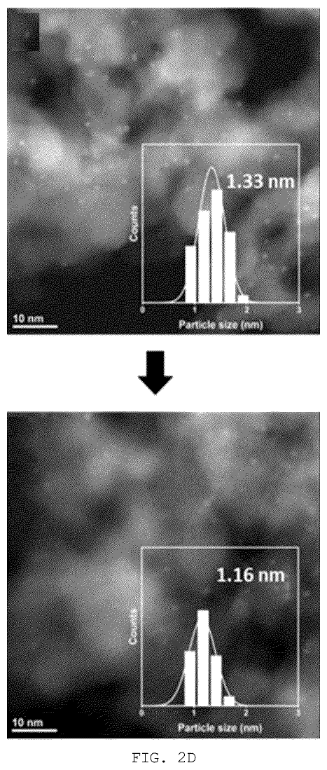

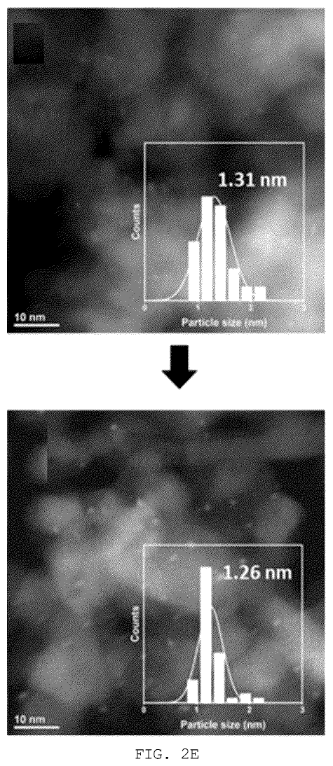

FIG. 1A shows an image of a synthesized catalyst composite of the present disclosure; FIGS. 2A to 2E show TEM images of Comparative Examples 1 (A), Comparative Examples 2 (B), Comparative Examples 3 (C), and Examples 1 (D) and Examples 2 (E) of the present disclosure.

The present disclosure provides a catalyst composite, the catalyst composite comprising: a support comprising a metal oxide doped with phosphorus(P); and a catalyst comprising a platinum group nanoparticle and sulfur(S) and supported on the support, wherein the platinum group nanoparticle may comprise a platinum group element, and the sulfur(S) may be doped on a part or all of a surface of the platinum group nanoparticle.

In addition, the doped phosphorus may be located at an acidic site of the metal oxide.

In addition, the phosphorus may be doped onto the metal oxide in the form of a phosphate group.

In addition, the support may be an alumina support of theta phase.

In addition, the support may comprise at least one selected from the group consisting of aluminum (Al) foam, aluminum (Al) mesh, nickel (Ni) foam, nickel mesh, copper (Cu) foam, copper mesh, titanium (Ti) foam, titanium mesh, graphene foam, graphene mesh, carbon paper, carbon felt and carbon foam, preferably aluminum (Al) foam or aluminum (Al) mesh.

In addition, the platinum group element may be a partially positively charged due to the sulfur.

In addition, the phosphate group may suppress the doping of the sulfur onto the metal oxide.

In addition, the metal oxide may comprise at least one selected from the group consisting of alumina (Al₂O₃), cerium oxide (CeO₂), magnesium oxide (MgO), carbon composite (C), silica (SiO₂) and titania (TiO₂).

In addition, the metal oxide may comprise a chloride series or nitrate series compound.

In addition, the catalyst composite may comprise 0.01 to 2 parts by weight, preferably 0.3 to 1.5 parts by weight, more preferably 0.8 to 1.0 parts by weight of the phosphorus based on 100 parts by weight of the metal oxide. When the catalyst composite comprises less than 0.01 parts by weight of the phosphorus based on 100 parts by weight of the metal oxide, the amount of phosphate doped is insufficient, which is undesirable. When the catalyst composite comprises more than 2 parts by weight, the amount of phosphate doped is large, which may interfere with the adsorption of the precious metal and sulfur, which is undesirable.

In addition, the platinum group element may comprise at least one selected from the group consisting of Pt, Pd, Ru, Rh, Os and Ir, preferably at least one selected from the group consisting of Pt, Pd and Ru, more preferably at least one selected from the group consisting of Pt and Pd.

In addition, the size of the platinum group nanoparticle may be in a range of 0.5 to 10 nm. When the size of the platinum group nanoparticles is less than 0.5 nm, side reactions increase during the hydrogen extraction reaction, which reduces the stability of the catalyst, which is undesirable. When the size exceeds 10 nm, the number of active site decreases, which reduces the efficiency of the catalyst, which is undesirable.

In addition, the catalyst composite may comprise 0.1 to 2 parts by weight, preferably 0.3 to 1 part by weight of the platinum group nanoparticle based on 100 parts by weight of the metal oxide. When the catalyst composite comprises less than 0.1 part by weight of the platinum group nanoparticles based on 100 parts by weight of the metal oxide, the size of the metal particles becomes very small and the possibility of occurrence of the side reaction increases, which is undesirable. When it comprises more than 2 parts by weight, the size of the metal particles becomes very large, which is undesirable because the number of active site decreases.

In addition, the catalyst composite may comprise 0.01 to 0.5 parts by weight of the sulfur, preferably 0.05 to 0.3 parts by weight, based on 100 parts by weight of the metal oxide. When the catalyst composite comprises less than 0.01 parts by weight of sulfur based on 100 parts by weight of the metal oxide, it is undesirable because it fails to impart a partial positive charge state to the platinum group nanoparticles. When it comprises more than 0.5 parts by weight, it is undesirable because the poisoning phenomenon of the platinum group nanoparticles significantly increases.

In addition, the catalyst composite may be used to extract hydrogen by dehydrogenating an organic hydrogen carrier.

Another aspect of the present disclosure provides a method of preparing a catalyst composite, the method comprising: (a) stirring a mixture comprising a metal oxide, a phosphorus(P) precursor, and a first solvent and drying; (b) preparing a support comprising a phosphorus(P)-doped metal oxide by heat-treating the resultant of step (a) thus preparing a support comprising a phosphorus(P)-doped metal oxide; (c) stirring a mixture comprising the support, a platinum group element precursor, and a second solvent and drying; (d) supporting platinum group nanoparticle comprising a platinum group element on the support by heat-treating the resultant of step (c); (e) stirring a mixture comprising the support on which the platinum group nanoparticles are supported, a sulfur precursor, and a third solvent and drying; and (f) preparing a catalyst composite by heat-treating the resultant of step (e).

In addition, the catalyst composite may comprise a support comprising a metal oxide doped with phosphorus(P); and a catalyst comprising platinum group nanoparticle and sulfur(S) and supported on the support, wherein the platinum group nanoparticle comprises a platinum group element, and the sulfur(S) is doped onto a part or all of a surface of the platinum group nanoparticle.

In addition, the heat treatments of steps (b), (d) and (f) may be carried out at a temperature range of 400 to 600° C. respectively. When the heat treatment is carried out at a temperature lower than 400° C., the size of the platinum group nanoparticles becomes small, which is undesirable. When it is carried out at a temperature higher than 600° C., the size of the platinum group nanoparticles becomes large, which is undesirable.

In addition, the first solvent, the second solvent, and the third solvent may comprise water respectively.

In addition, the phosphorus precursor may comprise at least one selected from the group consisting of (NH₄)₂HPO₄, NH₄H₂PO₄, phosphoric acid (H₃PO₄), phytic acid (C₆H₁₈O₂₄P₆), phosphine (PH₃), teriethoxyphosphine (C₆H₁₅O₃P) and triphenylphosphine ((C₆H₅)₃P), the platinum group element precursor comprises at least one selected from the group consisting of H₂PtCl₆, Pt(NO₃)₂, Pt(NH₃)₂ (NO₂)₂ and Pt(NH₃)₄ (OH)₂ and the sulfur precursor comprises at least one selected from the group consisting of (NH₄)₂SO₄, sulfuric acid (H₂SO₄), thiourea ((NH₂)₂CS), thioamide, hydrogen sulfide (H₂S) and sodium thiosulfate (Na₂S₂O₃).

In addition, the platinum group element precursor may comprise a chloride series or a nitrate series compound.

In addition, the platinum group element precursor may comprise at least one selected from the group consisting of PtCl₃, PtCl₃·XH₂O, PtCl₃·3H₂O, [Pt(NH₃)₆]Cl₂, Pt₃(CO) 12, [Pt(CO)₃Cl₂]₂, C₁₆H₂O₂Pt, C₁₈H₂₆Pt, Pt(NO)(NO₃)ₓ(OH)_{y}(wherein x+y=3), I₃Pt, Pt(C₅H₇O₂)₃, K₄Pt(CN)·xH₂O, PtO₂·xH₂O, PtO₂, KPtO₄ and K₂PtCl₆.

In addition, the alumina support may be the alumina support of theta phase.

In addition, the support may comprise at least one selected from the group consisting of aluminum (Al) foam, aluminum (Al) mesh, nickel (Ni) foam, nickel mesh, copper (Cu) foam, copper mesh, titanium (Ti) foam, titanium mesh, graphene (Graphene) foam, graphene mesh, carbon paper, carbon felt and carbon foam, preferably aluminum (Al) foam or aluminum (Al) mesh.

Another aspect of the present disclosure provides a method of extracting hydrogen, the method comprising: producing a compound represented by structural formula 2 and hydrogen by using a compound represented by structural formula 1 and a catalyst composite, as in reaction scheme 1.

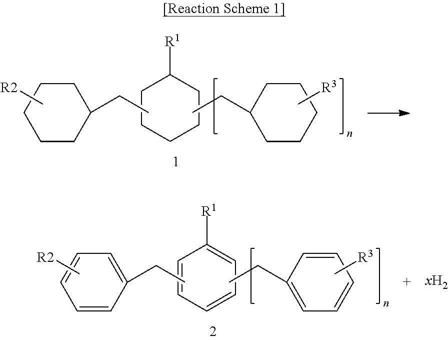

in the reaction scheme 1, R¹ is a hydrogen atom or a C1 to C3 alkyl group, R² is a hydrogen atom or a C1 to C3 alkyl group, R³ is a hydrogen atom or a C1 to C3 alkyl group, n is any one of integers 0 to 2, and x is any one of integers 6 to 12.

In addition, in the above reaction scheme 1, R¹ is a methyl group, R² is a hydrogen atom, R³ is a hydrogen atom, n is any one of integers 0 to 2, and x can be any one of integers 6 to 12.

In addition, the catalyst composite may comprise a support comprising a metal oxide doped with phosphorus(P); and a catalyst comprising platinum group nanoparticle and sulfur(S) and supported on the support, wherein the platinum group nanoparticle comprises a platinum group element, and the sulfur(S) may be doped on a part or all of a surface of the platinum group nanoparticle.

FIG. 1B shows a schematic diagram of a thermochemical liquid organic hydrogen carrier hydrogen extraction system of the present disclosure.

Referring to FIG. 1B, the present disclosure provides a thermochemical liquid organic hydrogen carrier hydrogen extraction system 10 comprising a tank 110 storing a toluene derivative, a fluid supply unit 100 supplying a fluid comprising a toluene derivative to a reactor 210; and a reaction unit 200 comprising the reactor 210 and the catalyst composite positioned inside the reactor 210 and extracting hydrogen from the toluene derivative.

In addition, the hydrogen extraction system 10 may additionally comprise a component analysis unit 300 comprising a component analyzer 310; and a separation unit 400.

In addition, the reaction unit 200 may additionally comprise a stirrer, a chiller, a water level sensor, a temperature controller (TC), and an exhaust unit (Vent).

Referring to FIG. 1B, in thermochemical liquid organic hydrogen carrier hydrogen extraction reaction system of the present disclosure, the catalyst is positioned in the center of a ½″ stainless reactor to proceed with the reaction, feed reactant utilizes a material obtained by hydrogenating heat transfer oil Marlotherm L H, the gas generated after the reaction is sent to GC-TCD using a quantitative pump and measured, and the liquid generated after the reaction is obtained using a distillation tower and measured through GC-FID.

The present disclosure relates to a platinum catalyst comprising sulfur and phosphorus for high-purity hydrogen extraction from a liquid organic hydrogen carrier and a method for producing the same, and relates to the synthesis of a platinum-based catalyst on a structured support, utilization thereof as a catalyst for thermochemical hydrogen extraction based on a liquid organic hydrogen carrier, and a catalyst for high-purity hydrogen extraction.

The present disclosure relates to a liquid organic hydrogen carrier decomposition hydrogen extraction system, production of a thermochemical alkane decomposition hydrogen production catalyst, catalyst production technology, low-precious-metal content catalyst technology, structured (bead) support, and commercialization catalyst technology. It is expected that it can be applied to a thermochemical liquid organic hydrogen carrier decomposition hydrogen extraction system and a hydrogen storage and hydrogen extraction continuous reactor, and in the future, it can be applied to a catalyst (hydrogen production catalyst) used in a thermochemical liquid organic hydrogen carrier decomposition plant, a thermochemical hydrogen generation device (hydrogen production system), hydrogen production through installation in an on-board mobility, and power production through fuel cell supply (device operation), thermochemical liquid organic hydrogen carrier reactor manufacturing, and structure catalyst manufacturing.

The present disclosure increases the efficiency of hydrogen extraction by doping phosphorus into a support to increase the dispersion of platinum particles. This increases the dispersion of platinum particles and at the same time provides stability to the catalyst, so that sintering phenomenon can be suppressed.

In addition, when sulfur is doped into a platinum catalyst using a phosphorus-doped support of the present disclosure, sulfur can be selectively adsorbed to the platinum particles. This makes it possible to control platinum particles even with a very small amount of sulfur, and maximize the stability of the catalyst. Therefore, the platinum loading amount can be minimized.

In addition, it can exhibit high activity under a reaction temperature of 320° C. and can improve the stability of the catalyst. Through this, it can be an indicator of the development of a catalyst with long-term durability.

The present disclosure preferably makes a phosphorus-doped support by adding (NH₄)₂HPO₄ to the Al₂O₃ support of theta phase. After that, platinum is dispersed through the addition of H₂PtCl₆ (0.5 wt % relative to the weight of the support), and then sulfur is selectively adsorbed on the platinum through the addition of (NH₄)₂SO₄. For each process, calcination is carried out at 500° C. for 3 hours under air conditions.

The present disclosure is not limited to the above preferred examples, and various metals and alkali metals can be supported together on the catalyst. In addition, it is possible to provide economic feasibility to the synthesis of the catalyst by using the simplest synthesis method (impregnation method).

### EXAMPLES

Hereinafter, the examples of the present disclosure will be described. However, the examples are for illustrative purposes, and the scope of the present disclosure is not limited by the examples.

### Example: Preparation of Catalyst by Wet-Impregnation Method

### Example 1: Preparation of Catalyst with Platinum Doped with Sulfur(S), Support Doped with Phosphorus(P), and Phosphorus Content of 0.5 wt % (SPt/0.5 PA)

### Preparation of Precursor Solution and Support

A precursor (Ammonium phosphate basic) in an amount of 0.9 wt % based on the weight of the support was added into distilled water (3rd, 17˜18.2 MΩ·cm). 10 g of theta phase alumina support was added to the solution and stirred at room temperature for more than 2 hours. The stirred support was collected after drying in an oven at 100° C. for 12 hours. The dried support was calcined at 500° C. for 3 hours in an air atmosphere.

### Platinum Supporting and Sulfur Doping

The platinum content of the above-mentioned manufactured support was made to be 0.5 wt % relative to the support. At this time, the support was stirred in a solution mixed with a platinum precursor (chloroplatanic acid) at room temperature for 2 hours or more. The stirred catalyst was collected after drying in an oven at 100° C. for 12 hours, and the dried catalyst was calcined at 500° C. for 3 hours in an air atmosphere.

The sulfur content of the catalyst was made to be 0.1 wt % relative to the support. At this time, the support was stirred in a solution mixed with a sulfur precursor (ammonium sulfate) at room temperature for 2 hours or more. The stirred catalyst was collected after drying in an oven at 100° C. for 12 hours, and the dried catalyst was calcined at 500° C. for 3 hours in an air atmosphere to dope platinum with sulfur(S) and to dope the support with phosphorus(P), thereby producing a SPt/0.5 PA catalyst comprising 0.5 wt % of platinum relative to the alumina support.

### Example 2:0.9 wt % Phosphorus Content (SPt/0.9 PA)

A SPt/0.9 PA catalyst was prepared in the same manner as Example 1, except that the phosphorus content in the prepared support was made to be 0.9 wt % (P loading 0.888 wt %) instead of 0.5 wt %.

### Comparative Example

### Comparative Example 1: Catalyst without Sulfur Doping on Platinum and without Phosphorus Doping on the Support (Pt/A)

A platinum/alumina catalyst (Pt/Al₂O₃) was prepared in the same manner as in Example 1, except that the platinum was not doped with sulfur and the support was not doped with phosphorus.

### Comparative Example 2: Catalyst without Sulfur Doping on Platinum and with Phosphorus (P) Doping on Support and a Phosphorus Content of 0.5 wt % (Pt/0.5 PA)

A Pt/0.5 PA catalyst (P loading 0.433 wt %) was prepared in the same manner as in Example 1, except that the platinum was not doped with sulfur.

### Comparative Example 3: Catalyst without Sulfur Doping on Platinum and with Phosphorus (P) Doping on Support, and P Content of 0.9 wt % (Pt/0.9 PA)

A Pt/0.9 PA catalyst was prepared in the same manner as in Example 1, except that the platinum was not doped with sulfur and the support had a phosphorus content of 0.9 wt % (P loading 0.866 wt %).

### Comparative Example 4: Catalyst with Sulfur Doping on Platinum, without P Doping on Support (SPt/A)

A SPt/A catalyst was prepared in the same manner as in Example 1, except that the platinum was doped with sulfur and the support was not doped with phosphorus

Table 1 below summarizes Comparative Examples 1 to 4 and Examples 1 and 2 of the present disclosure.

TABLE 1

| | | | | Phosphorous |
|---|---|---|---|---|
| | | Doped | Doped | content(wt |
| category | Code | sulfur | phosphorus | %) |
| Examples 1 | SPt/0.5PA | ∘ | ∘ | 0.459 |
| Examples 2 | SPt/0.9PA | ∘ | ∘ | 0.888 |
| comparative example 1 | Pt/A | x | x | — |
| comparative example 2 | Pt/0.5PA | x | ∘ | 0.433 |
| comparative example 3 | Pt/0.9PA | x | ∘ | 0.866 |
| comparative example 4 | SPt/A | ∘ | x | — |

### Test Example

### Test Example 1: Catalyst Composition and Property Analysis Data (ICP-OES Analysis)

The catalyst composition and property analysis data are shown in Table 2 below.

TABLE 2

| | | Pt | P | S | Surface | Pore | Pore | |
|---|---|---|---|---|---|---|---|---|
| catalyst | code | loading(wt %) | loading(wt %) | loading(wt %) | area(m²/g) | volume(cm³/g) | diameter(nm) | Dispersion(%) |
| comparative | Pt/A | 0.498 | — | — | 76.39 | 0.34 | 17.83 | 47.64 |
| example 1 | | | | | | | | |
| comparative | Pt/0.5PA | 0.508 | 0.433 | — | 75.18 | 0.33 | 17.68 | 55.38 |
| example 2 | | | | | | | | |
| comparative | Pt/0.9PA | 0.478 | 0.866 | — | 76.87 | 0.33 | 17.05 | 54.58 |
| example 3 | | | | | | | | |
| Example 1 | SPt/0.5PA | 0.437 | 0.459 | 0.113 | 76.99 | 0.33 | 17.20 | 44.23 |
| Example 2 | SPt/0.9PA | 0.419 | 0.888 | 0.114 | 76.88 | 0.33 | 17.03 | 36.88 |

According to Table 2, the catalyst was prepared by minimizing the platinum loading amount, and the results of quantitative analysis of platinum, phosphorus, and sulfur were obtained, which confirmed that the economic feasibility of the catalyst could be secured. In addition, the dispersion of platinum was measured to identify the active site that could be utilized.

### Test Example 2: Catalyst Characteristics Analysis (TEM Analysis)

FIGS. 2A to 2E show TEM images of Comparative Example 1 (A), Comparative Example 2 (B), Comparative Example 3 (C), and Example 1 (D) and Example 2 (E) of the present disclosure. The size of the Pt metal lump supported in the TEM image is approximately 1 to 2 nm.

Referring to FIGS. 2A to 2E, the nanoparticle size of the platinum particles on the catalyst was measured. This allowed the platinum particles supported on the catalyst to be identified and the dispersion to be primarily confirmed. In addition, it was found that the sintering phenomenon of the platinum particles did not occur on the support doped with phosphorus. In addition, it was confirmed that the dispersion increased. In addition, there was no significant change in the shape or size of the particles after sulfur doping. This indicates that the decrease in dispersion in the above physical property analysis was due to sulfur poisoning rather than sintering and loss of platinum particles.

### Test Example 3: Catalytic Activity Evaluation

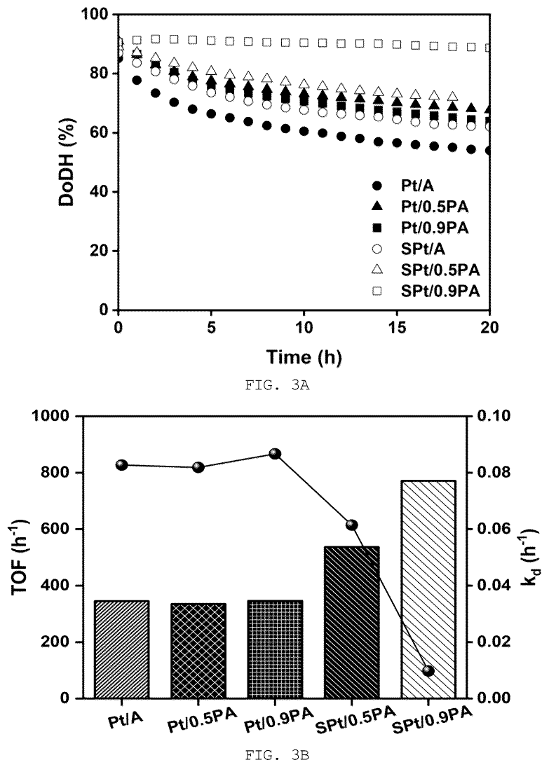

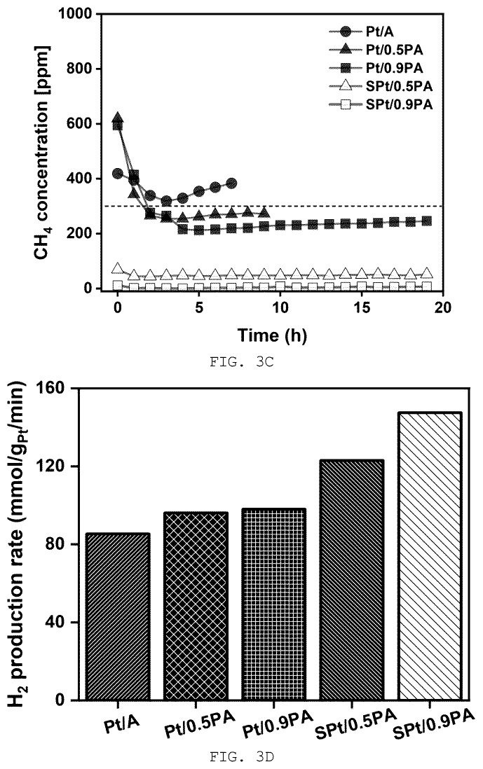

FIG. 3A shows the hydrogen extraction efficiency of Comparative Examples 1 to 4 and Examples 1 and 2 of the present disclosure, FIG. 3B shows the turnover frequency and deactivation rate of Comparative Examples 1 to 3 and Examples 1 and 2 of the present disclosure, FIG. 3C shows the hydrogen purity of Comparative Examples 1 to 3 and Examples 1 and 2 of the present disclosure, FIG. 3D shows the H₂-generation rate of Comparative Examples 1 to 3 and Examples 1 and 2 of the present disclosure.

Referring to FIGS. 3A to 3D, it was confirmed that Example 2 had the highest catalyst stability, TOF, hydrogen purity, and hydrogen production volume.

### Test Example 4: H₂-TPR Spectrum Analysis

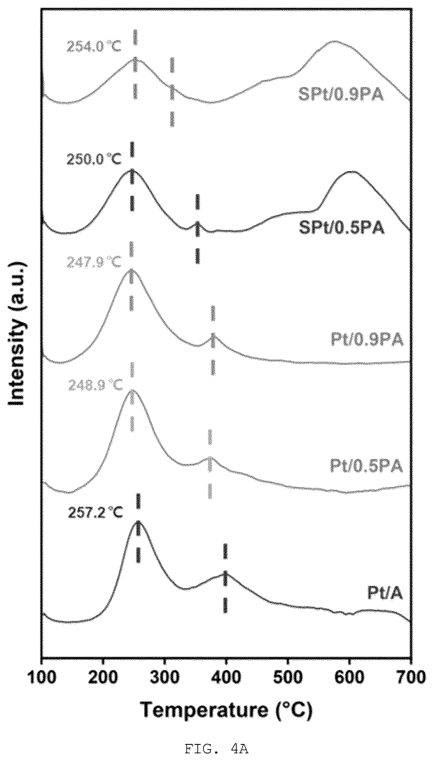

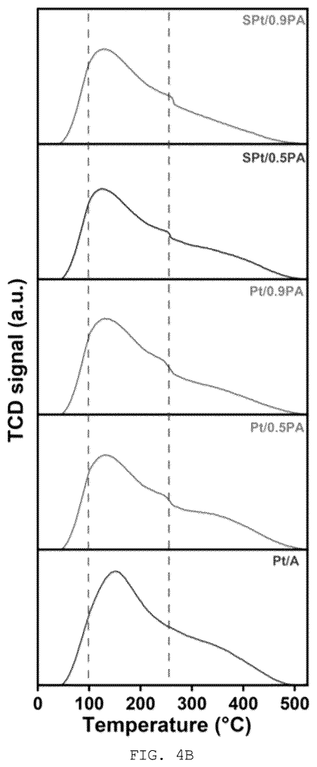

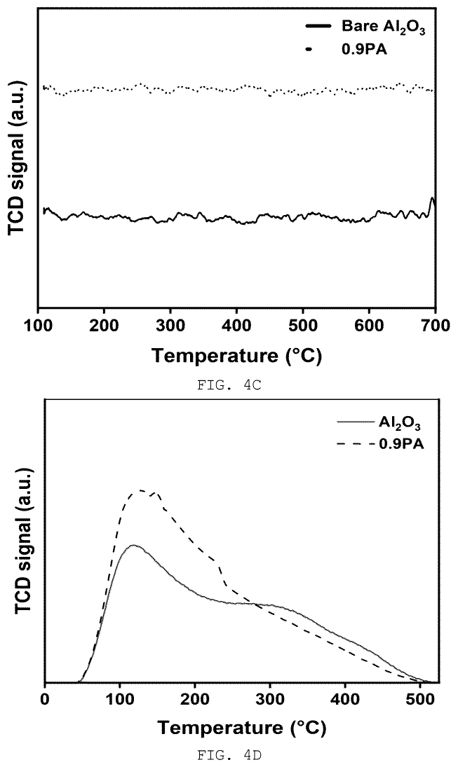

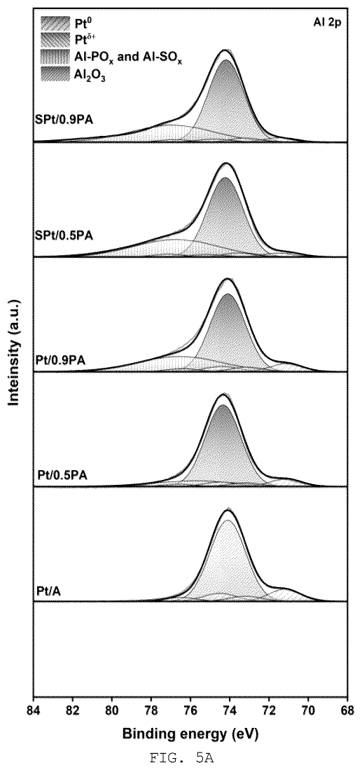

FIGS. 4A and 4C show the H₂-TPR spectra of bare Al₂O₃, 0.9P—Al₂O₃, Comparative Examples 1 to 3 and Examples 1 and 2 of the present disclosure.

Referring to FIGS. 4A and 4C, it can be seen that phosphate is adsorbed to the strong acidic site of the metal oxide in Comparative Examples 2 and 3, and in the case of Example 2, it can be confirmed that platinum group nanoparticles exhibiting partial positive charge are generated due to the interaction between phosphorus and sulfur. In particular, the reduction peak observed in the temperature range of approximately 200 to 450° C. is due to the decrease of Pt nanoparticles. In addition, since no peaks were observed in the bare support and PA, it can be confirmed that the two peaks are completely related to the Pt nanoparticles.

### Test Example 5: NH₃ desorption amount analysis of the synthesized catalyst in NH₃-TPD Analysis

FIGS. 4B and 4D show the NH₃-TPD spectra of bare Al₂O₃, 0.9P—Al₂O₃, Comparative Examples 1 to 3 and Examples 1 and 2 of the present disclosure.

Table 3 below summarizes and shows the NH₃ desorption amount analysis of the synthesized catalyst in NH₃-TPD analysis.

TABLE 3

| | | Acidic site(mmol/g) | | |
|---|---|---|---|---|
| | | | Medium & | |
| catalyst | Code | Weak | Strong | Total |
| comparative | Pt/A | 0.199 | 0.086 | 0.285 |
| example 1 | | | | |
| comparative | Pt/0.5PA | 0.179 | 0.080 | 0.259 |
| example 2 | | | | |
| comparative | Pt/0.9PA | 0.178 | 0.061 | 0.249 |
| example 3 | | | | |
| Examples 1 | SPt/0.5PA | 0.167 | 0.068 | 0.235 |
| Examples 2 | SPt/0.9PA | 0.173 | 0.056 | 0.229 |

Referring to FIGS. 4B, 4D and Table 3, it can be confirmed that the acidic site decreases through phosphate doping, and acidic site of Pt/0.5 PA and Pt/0.9 PA decreases in compared to that of Pt/A. It can be confirmed that sulfur is selectively adsorbed to platinum group nanoparticles through the change in the amount of acidic site reduced depending on the amount of phosphate.

### Test Example 6: XPS Spectrum Analysis

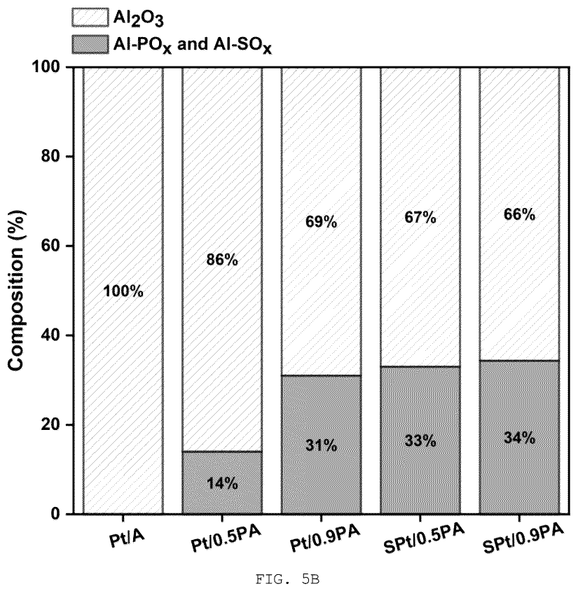

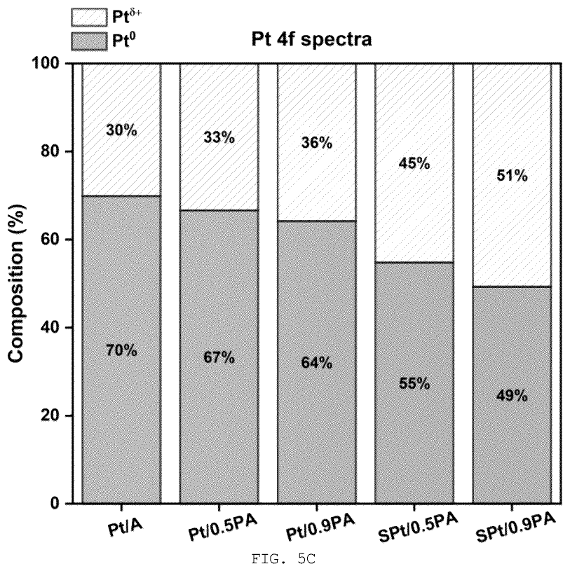

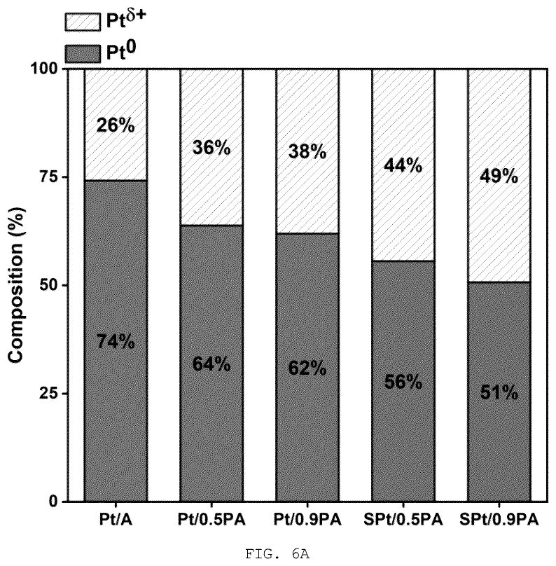

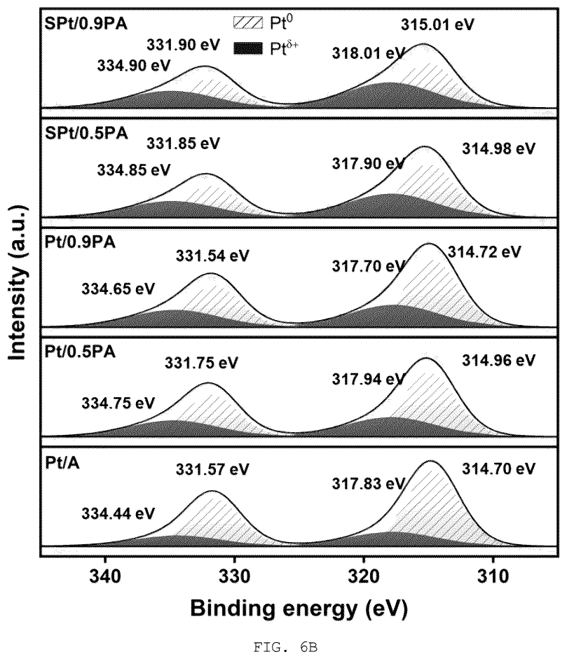

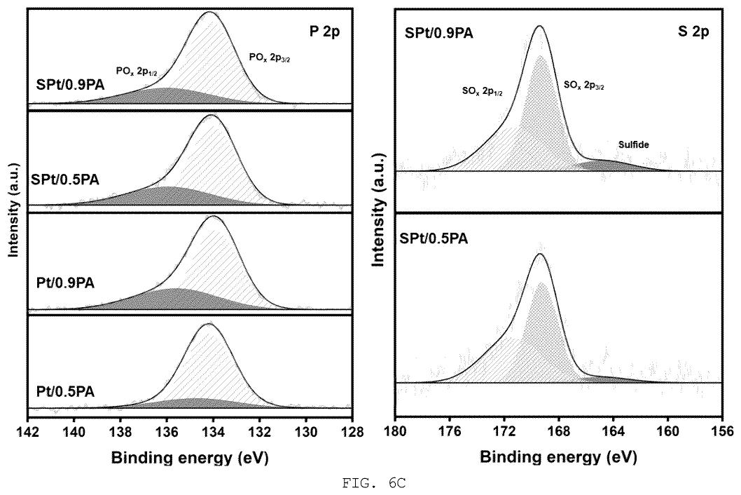

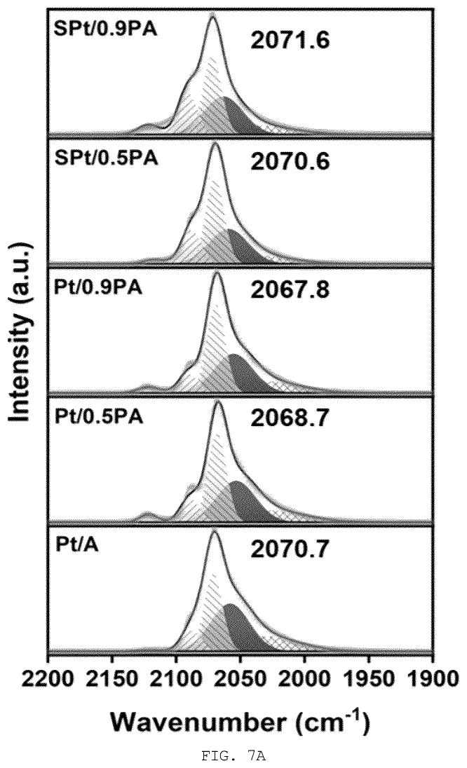

FIGS. 5A to 5C show the XPS spectra of Al 2p and Pt 4f for Comparative Examples 1 to 3 and Examples 1 and 2 of the present disclosure, FIG. 6A shows the ratio of Pt⁰ and Pt^{δ+} of Comparative Examples 1 to 3 and Examples 1 and 2 of the present disclosure, FIG. 6B shows the Pt 4d XPS spectra of Comparative Examples 1 to 3 and Examples 1 and 2 of the present disclosure, FIG. 6C shows the XPS spectra of P 2p (top) in the catalysts of Comparative Examples 2 and 3 and Examples 1 and 2 of the present disclosure and S 2p (bottom) in the catalysts of Examples 1 and 2 of the present disclosure.

Referring to FIGS. 5A to 5C, the composition of positively charged Pt atom further increased in Pt/0.9 PA due to sulfur impregnation compared to Pt/0.5 PA. It was confirmed that SPt/0.9 PA had the largest amount of ionic Pt atoms. In addition, it was confirmed that the Al 2p XPS spectrum provided additional evidence for the selective adsorption of sulfur.

Referring to FIGS. 6A to 6C, it was confirmed that in the case of Example 2, there were many platinum group nanoparticles with the most partial positive charge.

### Test Example 7: Quantification of Surface Pt Site of Prepared Catalyst Measured by CO-DRIFT

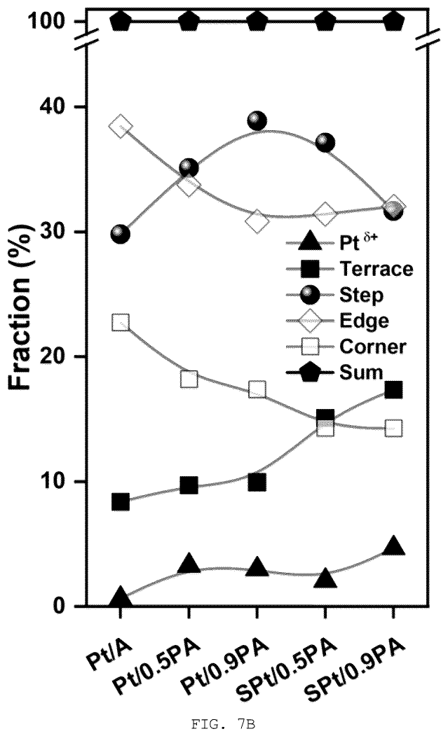

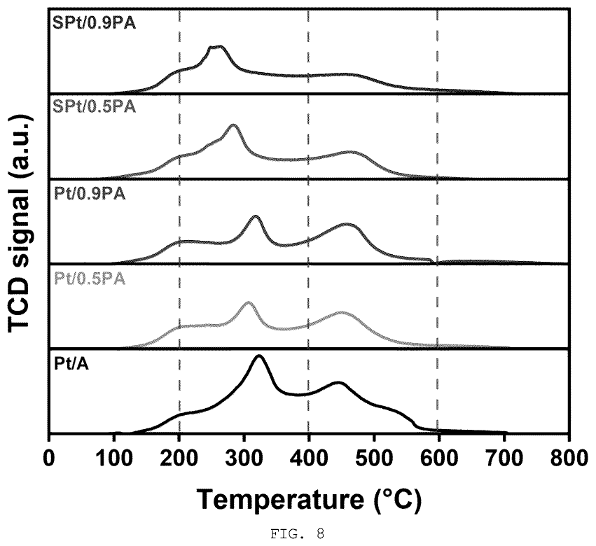

FIG. 7A shows the DRIFT spectra of CO adsorbed on the catalysts of Comparative Examples 1 to 3 and Examples 1 and 2 of the present disclosure, and FIG. 7B shows the quantification of Pt site types for the catalysts of Comparative Examples 1 to 3 and Examples 1 and 2 of the present disclosure.

Table 4 below summarizes the quantification of surface Pt site of prepared catalysts measured by CO-DRIFT.

TABLE 4

| | | Fraction of Pt surface | | | |
|---|---|---|---|---|---|
| | | PC | WC | UC | (PC + |
| Catalyst | Code | Pt(%) | Pt(%) | Pt(%) | WC)/UC |
| comparative | Pt/A | 0.64 | 8.38 | 90.98 | 0.10 |
| example 1 | | | | | |
| comparative | Pt/0.5PA | 3.27 | 9.71 | 87.02 | 0.15 |
| example 2 | | | | | |
| comparative | Pt/0.9PA | 2.98 | 9.94 | 87.08 | 0.15 |
| example 3 | | | | | |
| Examples 1 | SPt/0.5PA | 2.07 | 15.10 | 82.84 | 0.21 |
| Examples 2 | SPt/0.9PA | 4.67 | 17.36 | 77.97 | 0.28 |

Referring to FIGS. 7A, 7B and Table 4, it can be confirmed that sulfur is physically adsorbed the most in the low-coordination, high-activity site of platinum group nanoparticles in Example 2, and that platinum group nanoparticles with partial positive charge are the most numerous.

### Test Example 8: O₂-TPO Analysis

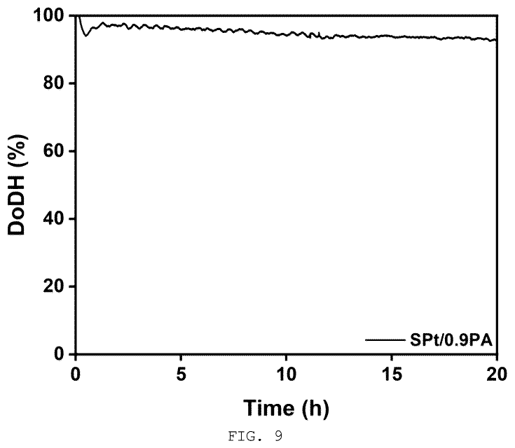

FIG. 8 shows the O₂-TPO analysis of Comparative Examples 1 to 3 and Examples 1 and 2 of the present disclosure.

Referring to FIG. 8, it can be confirmed that Example 2 has the highest resistance to carbon deposition.

### Test Example 9: Peak Positions of CO-DRIFT Analysis of Catalyst

Table 5 below shows the peak positions of CO-DRIFT analysis of catalysts.

TABLE 5

| | | Peak | Peak | Peak | Peak | Peak |
|---|---|---|---|---|---|---|
| Catalyst | CODE | 1(cm⁻¹) | 2(cm⁻¹) | 3(cm⁻¹) | 4(cm⁻¹) | 5(cm⁻¹) |
| comparative | Pt/A | 2122.20 | 2087.82 | 2071.25 | 2058.09 | 2033.50 |
| example 1 | | | | | | |
| comparative | Pt/0.5PA | 2121.70 | 2088.90 | 2068.15 | 2053.19 | 2028.46 |
| example 2 | | | | | | |
| comparative | Pt/0.9PA | 2121.18 | 2088.90 | 2068.10 | 2053.20 | 2028.50 |
| example 3 | | | | | | |
| Examples 1 | SPt/0.5PA | 2117.20 | 2088.10 | 2070.01 | 2059.18 | 2039.51 |
| Examples 2 | SPt/0.9PA | 2121.55 | 2091.05 | 2072.10 | 2063.10 | 2037.60 |

Referring to Table 5, it was confirmed that platinum group nanoparticles with partial positive charges of Example 2 were the most abundant.

### Test Example 10: Dehydrogenation of Methylcyclohexane (MCH)

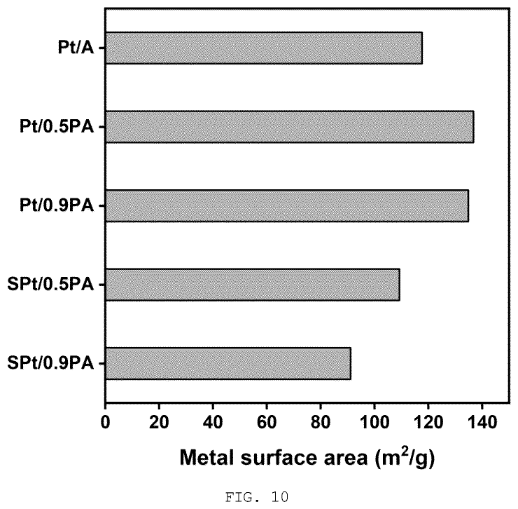

FIG. 9 shows the dehydrogenation of methylcyclohexane (MCH) of Example 2 of the present disclosure.

Referring to FIG. 9, it was confirmed that it was very efficient in the dehydrogenation reaction of general cyclic hydrocarbons.

### Test Example 11: Analysis of Metal Surface Area and ³¹P Solid-State NMR Spectroscopy of Catalyst

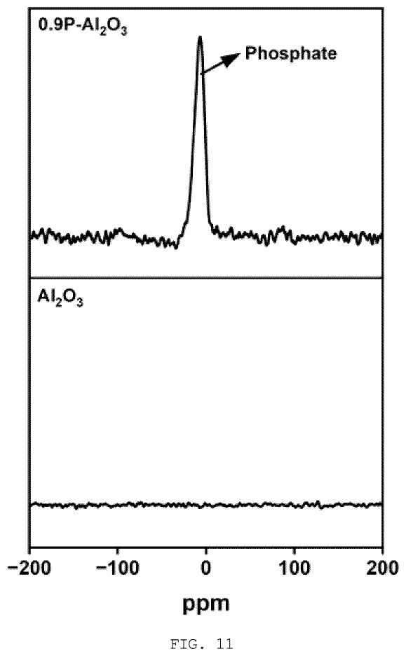

FIG. 10 shows the metal surface areas of catalysts of Comparative Examples 1 to 3 and Examples 1 and 2 of the present disclosure, and FIG. 11 shows the results of ³¹P solid-state NMR spectroscopy of alumina and 0.9P—Al₂O₃.

Referring to FIGS. 10 and 11, it was confirmed that the physicochemical properties of the synthesized catalyst did not change significantly except for metal dispersion. In particular, when Pt was loaded onto phosphorus-doped Al₂O₃, especially Pt/0.5 PA (Comparative Example 2) and Pt/0.9 PA (Comparative Example 3), the platinum dispersion increased compared to Pt/A (Comparative Example 1), and it was confirmed that phosphate was adsorbed on the acidic site of Al₂O₃ to form Al—O—POx or Al—POx. This site served as an anchoring site for metal during the impregnation step, thereby enhancing the dispersion. As a result, it was confirmed that the increase in metal dispersion was achieved through the interfacial bonding between Pt and phosphorus species.

The scope of the present disclosure is defined by the following claims rather than the above detailed description, and all changes or modifications derived from the meaning and scope of the claims and their equivalent concepts should be interpreted as falling into the scope of the present disclosure.

What is claimed is:

1. A catalyst composite, the catalyst composite comprising:
a support comprising a metal oxide doped with phosphorus(P); and
a catalyst comprising a platinum group nanoparticle and sulfur(S) and supported on the support,
wherein the platinum group nanoparticle comprises a platinum group element, and
the sulfur(S) is doped on a part or all of a surface of the platinum group nanoparticle.

2. The catalyst composite of claim 1, wherein the doped phosphorus is located at an acidic site of the metal oxide.

3. The catalyst composite of claim 1, wherein the phosphorus is doped onto the metal oxide in the form of a phosphate group.

4. The catalyst composite of claim 3, wherein the platinum group element is partially positively charged due to the sulfur.

5. The catalyst composite of claim 3, wherein the phosphate group suppresses the doping of the sulfur onto the metal oxide.

6. The catalyst composite of claim 1, wherein the metal oxide comprises at least one selected from the group consisting of alumina (Al₂O₃), cerium oxide (CeO₂), magnesium oxide (MgO), carbon composite (C), silica (SiO₂) and titania (TiO₂).

7. The catalyst composite of claim 1, wherein the catalyst composite comprises 0.01 to 2 parts by weight of the phosphorus based on 100 parts by weight of the metal oxide.

8. The catalyst composite of claim 1, wherein the platinum group element comprises at least one selected from the group consisting of Pt, Pd, Ru, Rh, Os and Ir.

9. The catalyst composite of claim 1, wherein the size of the platinum group nanoparticle is in a range of 0.5 to 10 nm.

10. The catalyst composite of claim 1, wherein the catalyst composite comprises 0.1 to 2 parts by weight of the platinum group nanoparticle based on 100 parts by weight of the metal oxide.

11. The catalyst composite of claim 1, wherein the catalyst composite comprises 0.01 to 0.5 parts by weight of the sulfur based on 100 parts by weight of the metal oxide.

12. The catalyst composite of claim 1, wherein the catalyst composite is used to extract hydrogen by dehydrogenating an organic hydrogen carrier.

13. A method of preparing a catalyst composite, the method comprising:
(a) stirring a mixture comprising a metal oxide, a phosphorus(P) precursor, and a first solvent and drying;
(b) preparing a support comprising a phosphorus(P)-doped metal oxide by heat-treating the resultant of step (a);
(c) stirring a mixture comprising the support, a platinum group element precursor, and a second solvent and drying;
(d) supporting platinum group nanoparticle comprising a platinum group element on the support by heat-treating the resultant of step (c);
(e) stirring a mixture comprising the support on which the platinum group nanoparticle are supported, a sulfur precursor, and a third solvent and drying; and
(f) preparing a catalyst composite by heat-treating the resultant of step (e).

14. The method of claim 13, wherein the catalyst composite comprises a support comprising a metal oxide doped with phosphorus(P); and a catalyst comprising platinum group nanoparticle and sulfur(S) and supported on the support,
wherein the platinum group nanoparticle comprises a platinum group element, and
the sulfur(S) is doped onto a part or all of a surface of the platinum group nanoparticle.

15. The method of claim 13, wherein the heat treatments of steps (b), (d) and (f) are carried out at a temperature range of 400 to 600° C. respectively.

16. The method of claim 13, wherein the first solvent, the second solvent, and the third solvent comprise water respectively.

17. The method of claim 13, wherein the phosphorus precursor comprises at least one selected from the group consisting of (NH₄)₂HPO₄, NH₄H₂PO₄, phosphoric acid (H₃PO₄), phytic acid (C6H₁₈O₂₄P₆), phosphine (PH₃), teriethoxyphosphine (C6H₁₅O₃P) and triphenylphosphine ((C6H₅)₃P), the platinum group element precursor comprises at least one selected from the group consisting of H₂PtCl₆, Pt(NO₃)₂, Pt(NH₃)₂(NO₂)₂ and Pt(NH₃)₄(OH)₂ and the sulfur precursor comprises at least one selected from the group consisting of (NH₄)₂SO₄, sulfuric acid (H₂SO₄), thiourea ((NH₂)₂CS), thioamide, hydrogen sulfide (H₂S) and sodium thiosulfate (Na₂S₂O₃).

18. A method of extracting hydrogen, the method comprising: producing a compound represented by structural formula 2 and hydrogen by using a compound represented by structural formula 1 and a catalyst composite, as in reaction scheme 1 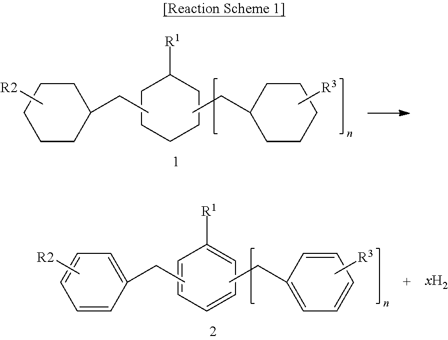
in the reaction scheme 1, R¹ is a hydrogen atom or a C₁ to C₃ alkyl group, R² is a hydrogen atom or a C₁ to C₃ alkyl group, R³ is a hydrogen atom or a C₁ to C₃ alkyl group, n is any one of integers 0 to 2, and x is any one of integers 6 to 12.

19. The method of claim 18, wherein in the reaction scheme 1, R¹ is a methyl group, R² is a hydrogen atom, R³ is a hydrogen atom, n is any one of integers 0 to 2, and x is any one of integers 6 to 12.

20. The method of claim 18, wherein the catalyst composite comprises a support comprising a metal oxide doped with phosphorus(P); and a catalyst comprising platinum group nanoparticle and sulfur(S) and supported on the support,
wherein the platinum group nanoparticle comprises a platinum group element, and the sulfur(S) is doped on a part or all of a surface of the platinum group nanoparticle.
